(19) United States
(12) Patent Application Publication
(10) Pub. No.: US 2017/0156649 A1
(43) Pub. Date: Jun. 8, 2017

(54) SYSTEM AND METHOD FOR MEASURING HEMOGLOBIN LEVEL

(71) Applicant: Harsh S. Modi, Melbourne, FL (US)

(72) Inventors: Harsh S. Modi, Melbourne, FL (US); Jay Modi, Ahmedabad, IN (US)

(73) Assignee: Harsh S. Modi, Melbourne, FL (US)

(21) Appl. No.: 15/368,795

(22) Filed: Dec. 5, 2016

Related U.S. Application Data
(60) Provisional application No. 62/263,374, filed on Dec. 4, 2015.

(51) Int. Cl. A61B 5/145; A61B 5/0205; A61M 16/00; A61B 5/0295; A61B 5/00; A61B 5/1455; A61B 5/026

(52) U.S. Cl. CPC A61B 5/14546; A61B 5/14535; A61B 5/14552; A61B 5/0205; A61B 5/0261; A61B 5/0295; A61B 5/7282; A61M 16/0069; A61M 16/0051; A61B 5/02416

(57) ABSTRACT

A hemoglobin measuring device comprising a controller, a first oxygen sensor, a second oxygen sensor, a first volume flow meter, and a second volume flow meter. The controller is operable to receive data from each of the first and second oxygen sensors, the first and second volume flow meters, and a pulse oximeter associated with a patient. The controller is also operable to determine a hemoglobin level of the patient, defined as a determined hemoglobin level, based on the data received from the first and second oxygen sensors, the first and second volume flow meters, and the pulse oximeter.

### RELATED APPLICATIONS

This application claims the benefit under 35 U.S.C. §119(e) OF U.S. Provisional Patent Application Ser. No. 62/263,374 titled System and Method for Measuring Hemoglobin Level filed Dec. 4, 2015, the entire content of which is incorporated herein by reference.

### FIELD OF THE INVENTION

The present invention relates to systems and methods for in vivo measurement of hemoglobin, among other physiological statues and conditioners, without requiring a blood sample.

### BACKGROUND

The measurement of hemoglobin levels is an integral part of providing care to patients in many settings, including intensive care settings. Historical methods of obtaining hemoglobin levels have been by direct observation, e.g. taking a blood sample and measuring the hemoglobin in the blood. However, there are inherent risks involved in obtaining blood samples, especially where hemoglobin needs to be monitored on a continuing and ongoing basis, requiring either multiple blood draws or the use of a permanent intravenous catheter. Accordingly, there is a need in the art for a system for measuring hemoglobin that does not require the drawing of blood.

There are systems available that provide total hemoglobin measurements via sensors positioned on the patient where optical transmissivity of the vasculature can be measured, such as on the patient's finger. However, the accuracy of such systems is uncertain, particularly in regards to oxygen consumption, and thus there is a need in the art for a more accurate system for the measurement of hemoglobin.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

With the above in mind, embodiments of the present invention are related to a hemoglobin measuring device comprising a controller, a first oxygen sensor, a second oxygen sensor, a first volume flow meter, and a second volume flow meter. The controller may be operable to receive data from each of the first oxygen sensor, the first volume flow meter, the second oxygen sensor, the second volume flow meter, and a pulse oximeter associated with a patient. Additionally, the controller may be operable to determine a hemoglobin level of the patient, defined as a determined hemoglobin level, based on the data received from the first oxygen sensor, the first volume flow meter, the second oxygen sensor, the second volume flow the pulse oximeter.

In some embodiments, the controller may be operable to determine a measured inhalation oxygen from data received from the first oxygen sensor and the first volume flow meter. Additionally, the controller may be operable to determine a measured exhalation oxygen from data received from the second oxygen sensor and the second volume flow meter. Furthermore, the controller may be operable to determine each of a breath volume and a breath rate from data received from the first volume flow meter and the second volume flow meter. The controller may further be operable to determine an oxygen consumption level from the measured inhalation oxygen and the measured exhalation oxygen.

Furthermore, the controller may be operable to receive an oxygen saturation percentage from the pulse oximeter and to determine the hemoglobin level of the patient using the formula:

$$\frac{\begin{matrix}( measured inhalation oxygen - measured exhalation oxygen ) * \\ breath volume\end{matrix}}{oxygen saturation * 1.36} .$$

Additionally, the controller may be operable to determine a red blood cell (RBC) count by dividing the determined hemoglobin level by 2.89.

In some embodiments, the device may further comprise an end tidal carbon dioxide (ETCO2) sensor. The controller may be operable to receive an ETCO2 level from the ETCO2 sensor. Additionally, the controller may be operable to determine at least one of a pleth variability index, a perfusion index, blood velocity, blood volume, and cardiac output information based on at least one of the determined hemoglobin level, the ETCO2 level, the oxygen saturation percentage, the breath volume, the breath rate, the oxygen consumption level, and a gas exchange rate. Furthermore, the controller may be operable to determine an indication of at least one of acute respiratory distress syndrome (ARDS), sepsis, and septic shock from at least one of the determined hemoglobin level, the ETCO2 level, the oxygen saturation percentage, the breath volume, the breath rate, the oxygen consumption level, and the gas exchange rate.

In some embodiments, the device may further comprise an alarm. The controller may be operable to determine if data received from any of the first and second oxygen sensors and the first and second volume flow meters is outside a target range and to operate the alarm responsive to at least one of a sudden drop in the determined hemoglobin level, an indication of ARDS, an indication of sepsis, an indication of septic shock, and an indication of data received from any of the first and second oxygen sensors and the first and second volume flow meters is outside the target range.

In some embodiments, the device may further comprise a data interface operable to transmit data to a ventilator. The data interface may be operable to transmit at least one of hemoglobin level, oxygen saturation, red blood cell count, lung volume, breath rate, and breath volume for display on a monitor comprised by the ventilator. Furthermore, the controller may be operable to send instructions to cause the ventilator to change its operation responsive to the determined hemoglobin level.

In some embodiments, the controller may be operable to receive instructions from a user via a remote access device. Additionally, the controller may be operable to send instructions to cause the ventilator to change its operation responsive to the instruction received by the remote access device.

In some embodiments, the first oxygen sensor and first volume flow meter may transmit data to the controller via a first fluid data path, the second oxygen sensor and second volume flow meter transmit data to the controller via a second fluid data path.

In some embodiments, the device may further comprise an interface operable to receive patient statistics. The controller may be operable to receive patient statistics via the interface. Additionally, the controller may be operable to make a recommendation regarding at least one of a change to a ventilator setting and a disease diagnosis responsive to the patient statistics and the determined hemoglobin level.

### BRIEF DESCRIPTION OF THE DRAWINGS

FIG. 1 is a schematic diagram of a system for measuring hemoglobin according to an embodiment of the invention.

FIG. 2 is a flowchart illustrating a method according to an embodiment of the invention.

FIG. 3 is a schematic diagram of a system for measuring hemoglobin according to another embodiment of the invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a system and related methods for the determination of hemoglobin and red blood cell levels in vivo in a patient without requiring a blood sample from the patient. The system measures oxygen consumption during respiration utilizing oxygen sensors attached to a device that monitors and/or controls the patient's respiration. Specifically, the system measures oxygen concentration in air prior to and after respiration by the patient, yielding the patient's oxygen consumption. The system further measures the patient's pulse oximetry during respiration, yielding the patient's oxygen saturation. Furthermore, the system relates the patient's oxygen consumption to the patient's oxygen saturation to determine the hemoglobin level and red blood cell count of the patient. The system may further deliver the hemoglobin level and red blood cell count of the patient to an external device, such as a display device or a ventilator, such that the hemoglobin level and red blood cell count may prompt some action.

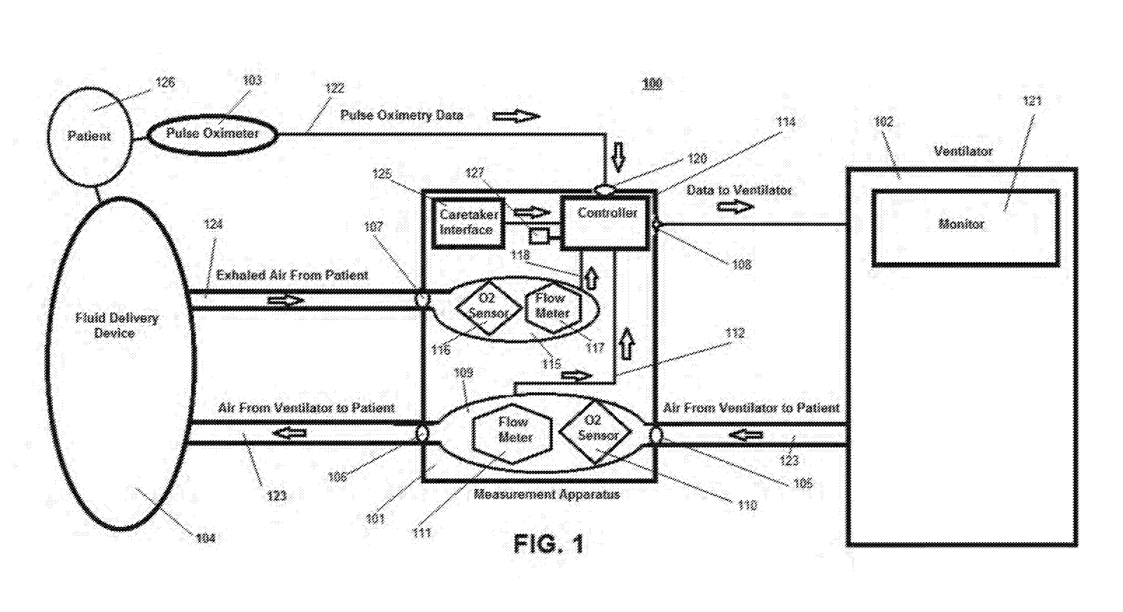

Referring now to FIG. 1, a schematic of a hemoglobin measuring system 100 according an embodiment of the invention is presented. The hemoglobin measuring system 100 may comp e a measurement apparatus 101, a ventilator 102, a pulse oximeter 103, and a fluid delivery device 104.

The measurement apparatus 101 may include a ventilator tubing interface 105, inhalation tubing interface 106, an exhalation tubing interface 107, a data interface 108, a pulse oximetry data interface 120, and a caretaker interface 125. The caretaker interface 125 may include a monitor and digital keypad configured so as to enter a patient's personal information including their gender, height, weight, and disease description. This information is sent via data path 119 connected at one end to the caretaker interface 125 and at another end to an internal controller 114 for processing. In some embodiments, the caretaker interface 125 may be a touchscreen.

Comprised by the measurement apparatus 101 and connected to the ventilator tubing interface 105 at one end and the inhalation tubing interface 106 at another end is a first fluid conversion member 109. The first fluid conversion member 109 may comprise a first oxygen sensor 110 configured to receive fluid from the ventilator 102 and measure the oxygen content of fluid received thereby. Connected to the first oxygen sensor 110 is a first volume flow meter 111 configured to measure the volume of fluid passing therethrough, and ergo to the patient, and deliver the output data in milliliters(mL). While particular units of measurement are recited herein, it is contemplated and included within the scope of the invention that any unit of measurement and/or system of measurement may be utilized. Connected to the first fluid conversion member 109 is a first fluid data path 112. The first fluid data path 112 is connected to a controller 114 and configured so as to deliver the measurement data from the first volume flow meter 111 to the controller 114 for processing.

Also comprised by the measurement apparatus 101 may be a second fluid conversion member 115. The second fluid conversion member 115 may comprise a second oxygen sensor 116 configured to receive fluid exhaled from a patient. Connected to the second oxygen sensor 116 is a second volume flow meter 117 configured to measure the volume of fluid passing therethrough and deliver the output data in milliliters (mL). Connected to the second fluid conversion member 115 is a second fluid data path 118. The second fluid data path 118 is connected to the controller 114 and configured so as to deliver the measurement data from the second volume flow meter 117 to the controller 114 for processing.

Although first and a second fluid conversion members 109, 115 as well as first and second oxygen sensors 110, 116, first and second volume flow meters 111, 117 and first and second fluid data paths 112, 118 are described herein, it is contemplated to be within the scope of the present invention that the functions performed by these components may be implemented by a single fluid conversion member, a single oxygen sensor, a single volume flow meter, and a single fluid data path located within the measurement apparatus 101. Moreover, the volume flow meter(s) may provide the breath volume of the patient.

The measurement apparatus 101 may be connected to the ventilator 102 via the data interface 108 and the ventilator tubing interface 105. Furthermore, the ventilator 102 may comprise a monitor 121 configured to display data from the measurement apparatus 101.

The measurement apparatus 101 may be connected to a pulse oximeter 103. The pulse oximeter 103 is attached to a patient at one end and utilizes an oximetry data path 122 to connect another end to the pulse oximeter data interface 120 on the measurement apparatus 101. The pulse oximeter data interface 120 connects to the controller 114 that is configured to process a patient's pulse oximetry data in conjunction with other data.

The measurement apparatus 101 may be connected to a fluid delivery device 104 via inhalation tubing 123 and exhalation tubing 124. Inhalation tubing 123 may be attached to the measurement apparatus 101 at the inhalation tubing interface 106. The inhalation tubing 123 is configured to act as a conduit for measured fluid delivered from the ventilator 102 to the fluid delivery device 104. The fluid delivery device 104 is configured to deliver the measured fluid to the patient for inhalation. The fluid delivery device 104 may also be connected to the measurement apparatus 101 via the exhalation tubing 124 attached to the fluid delivery device 104 on one end and the exhalation tubing interface 107 on another end. The exhalation tubing 124 is configured to act as a conduit for a patient's exhaled fluid that is to be measured by the second fluid conversion member 115, the second oxygen sensor 116, and the second volume flow meter 117.

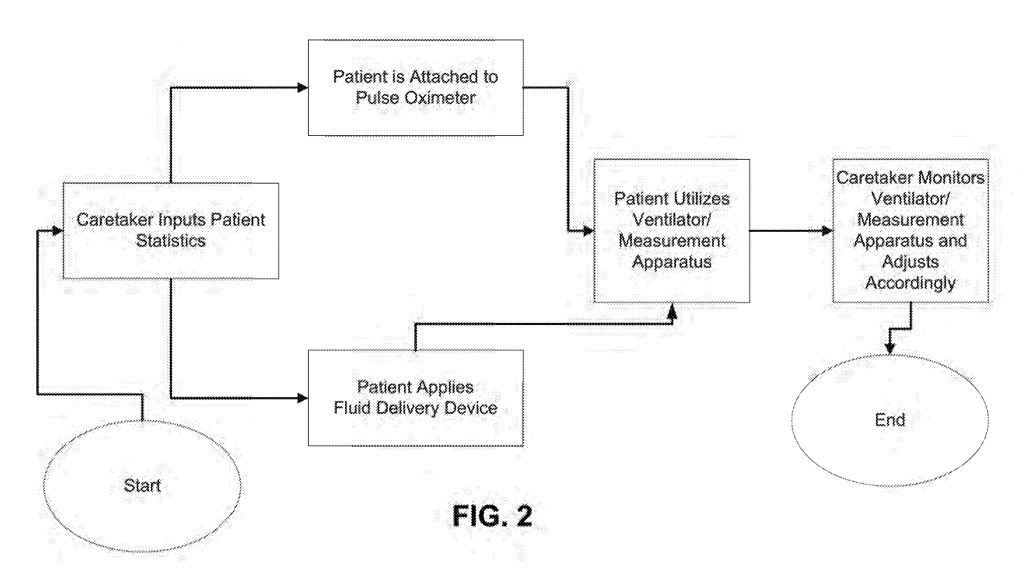

Referring now to FIGS. 1 and 2, methods of using the hemoglobin measuring system 100 will now be discussed. To begin the process, a caretaker types in a patient's statistics using the caretaker interface 125. Patient statistics may include a patient's gender, height, weight, and disease description. These statistics are stored in the controller 114 for use in conjunction with other data to be received. A patient 126 is then connected to a pulse oximeter 103 that delivers the patients pulse oximetry data to the measurement apparatus 101 controller 114 for processing. Simultaneously, a patient 126 utilizes the fluid delivery device 104 to inhale fluid provided by the ventilator 102 via the inhalation tubing 123. The fluid delivery device 104 may be, for example, a mask, direct tubing, tracheostomy tubing, or any device or means of delivering fluid such as air to the patient 126 from the measurement apparatus 101 and/or from the ventilator 102. The fluid delivery device 104 also receives exhaled fluid from the patient 126 and delivers the exhaled fluid to the measurement apparatus 101 via exhalation tubing 124.

The fluid provided to the patient 126 by the ventilator 102 travels from the ventilator 102 via inhalation tubing 123 to the measurement apparatus 101. The measurement apparatus 101 utilizes the first fluid conversion member 109 to measure the fluid content ultimately provided to the patient 126 and sends the measurement data to the controller 114 for processing. The first oxygen sensor 110 located within the first fluid conversion member 109 measures the oxygen content of the fluid to be provided to the patient 126. Similarly, the first volume flow meter 111 measures the volume of fluid passed therethrough and delivers the data to the controller 114. The oxygen content data of the air to be inhaled by the patient 126 is then sent by the first fluid conversion member 109 to the controller 114 for processing. The fluid then travels via inhalation tubing 123 to the fluid delivery device 104 and is inhaled by the patient.

When the patient exhales into the fluid delivery device 104 the exhaled fluid may be sent to the measurement apparatus 101 via the exhalation tubing 124. The measurement apparatus 101 utilizes the second fluid conversion member 115 to measure the fluid content of the patient's breath and sends the measurement data to the controller 114 for processing. The second oxygen sensor 116 located within the second fluid conversion member 115 measures the oxygen content of the patient's breath. Similarly, the second volume flow meter 117 measures the volume of fluid passed therethrough and delivers the data to the controller 114. The oxygen content data of the patient's breath is then sent by the second fluid conversion member 115 to the controller 114 in mL for processing. Based on the rate at which the controller 114 receives data from the first and second volume flow meters 111, 117, the controller 114 may be operable to determine a breath rate associated with the patient. Additionally, the controller 114 may be operable to determine a breath volume from the data received from the first and second volume flow meters 111, 117.

The controller 114 of the measurement apparatus 101 processes the patient's oxygen consumption data, defining an oxygen consumption level, and the patient's oxygen saturation data, defining an oxygen saturation percentage, to determine the patient's hemoglobin level, defining a determined hemoglobin level, and red blood cell (RBC) count. This is accomplished by the following formula:

((measured inhalation oxygen)−(measured exhalation oxygen)* breath volume)/(oxygen saturation* 1.36), Formula 1

The measured inhalation oxygen and the measured exhalation oxygen are percentages output by the oxygen sensors 110, 116. These percentages are multiplied by the breath volume output by the second volume flow meter 117 to determine how much oxygen has been consumed by a patient. As is known in the art, one gram of hemoglobin binds four molecules of oxygen yielding 1.36 mL at 100% saturation. Therefore the oxygen saturation data, also delivered as a percentage by the pulse oximeter 103, is multiplied by a 100% saturation at 1.36. By dividing the consumed oxygen level by the oxygen saturation level, the controller 114 is able to output a patient's hemoglobin level. Furthermore, once the hemoglobin level is established, the patient's red blood cell count (RBC) is determined by dividing the hemoglobin level by 2.89.

The measurement apparatus 101 may allow for automated control of the ventilator 102 output. The controller 114 may interface with the ventilator 102 and monitor 121 to display the patients real time hemoglobin level, oxygen saturation, red blood cell count (RBC), lung volume, breathing rate, breath volume, and potential disease diagnosis. Furthermore, the measurement apparatus 101 may be equipped with an alarm to alert a caretaker if there is a sudden drop in a patient's vital statistics such as hemoglobin and/or red blood cell count thereby indicating blood loss or other complication. Depending on whether the ventilator 102 is on a fully supported mode or assistive/weaning mode, the controller 114 is configured to make suggestions or directly make changes to the ventilator 102 settings and output. Based on the patient's response data, if the ventilator 102 is on a fully supported mode, the controller 114 is configured to make suggestions to the caretaker to adjust the ventilator 102 settings. If the ventilator is 102 on an assistive/weaning mode, the controller 114 is configured to automatically make corrective adjustments to the ventilator 102 settings accordingly.

In some embodiments, the hemoglobin measuring system 100 may be configured to permit remote control of settings to allow caretakers to remotely change a patient's fluid settings. This may be accomplished via a remote access device 127 located within the measurement apparatus that communicates with the controller 114. The remote control may result in dismissal of the alert, alert of medical professionals within the vicinity of the patient, alteration of the volume of fluid delivered to the patient, alteration of the oxygen content of fluid delivered to the patient, and alteration of the administration of medication to the patient.

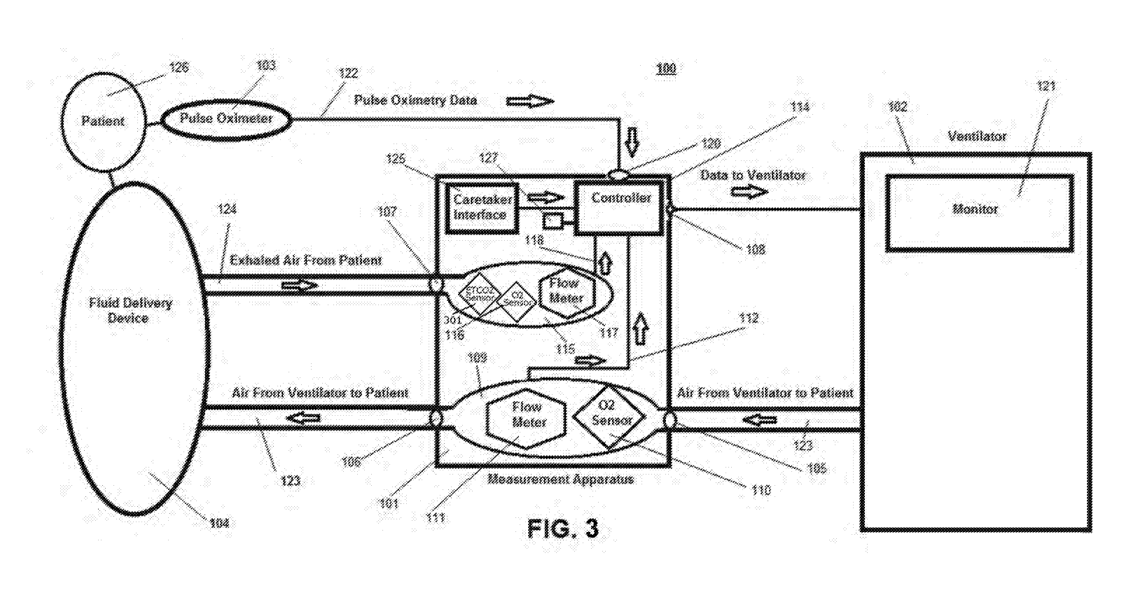

Referring now to FIG. 3, another embodiment of the present invention is presented. The measurement apparatus 101 of FIG. 3 is identical to the apparatus of FIG. 1, with the additional comprisal of an end tidal carbon dioxide (ETCO2) sensor 301 positioned in communication with the controller 114. The ECTO2 sensor 301 is operable to provide information regarding the ETCO2 measured from the exhalation tubing 124 and transmit such information to the controller 114 via the second fluid data path 118. The controller 114 may be operable to receive an indication of an ETCO2 level from the ETCO2 sensor 301. Additionally, the controller 114 may be operable to determine at least one of a pleth variability index, a perfusion index, blood velocity, blood volume, and cardiac output information based on at least one of the determined hemoglobin level, the ETCO2 level, the oxygen saturation percentage, the breath volume, the breath rate, the oxygen consumption level, and a gas exchange rate, the gas exchange rate being determined by the controller 114 from a comparison of the ETCO2 level and the oxygen consumption rate.

Furthermore, the controller 114 may be operable to determine an indication of at least one of acute respiratory distress syndrome (ARDS), sepsis, and septic shock from at least one of the determined hemoglobin level, the ETCO2 level, the oxygen saturation percentage, the breath volume, the breath rate, the oxygen consumption level, and the gas exchange rate.

As noted above, the measurement apparatus 101 may further comprise an alarm. The controller 114 may be operable to determine if data received from any of the first and second oxygen sensors 110, 116 and the first and second volume flow meters 111, 117 is outside a target range and to operate the alarm responsive to at least one of a sudden drop in the determined hemoglobin level, an indication of ARDS, an indication of sepsis, an indication of septic shock, and an indication of data received from any of the first and second oxygen sensors 110, 116 and the first and second volume flow meters 111, 117 is outside the target range.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawing and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

That which is claimed is:

1. A hemoglobin measuring device comprising:
a controller;
a first oxygen sensor;
a second oxygen sensor;
a first volume flow meter; and
a second volume flow meter;
wherein the controller is operable to receive data from each of the first oxygen sensor, the first volume flow meter, the second oxygen sensor, the second volume flow meter, and a pulse oximeter associated with a patient; and
wherein the controller is operable to determine a hemoglobin level of the patient, defined as a determined hemoglobin level, based on the data received from the first oxygen sensor, the first volume flow meter, the second oxygen sensor, the second volume flow meter, and the pulse oximeter.

2. The device according to claim 1 wherein:
the controller is operable to determine a measured inhalation oxygen from data received from the first oxygen sensor and the first volume flow meter;
the controller is operable to determine a measured exhalation oxygen from data received from the second oxygen sensor and the second volume flow meter;
the controller is operable to determine each of a breath volume and a breath rate from data received from the first volume flow meter and the second volume flow meter; and
the controller is operable to determine an oxygen consumption level from the measured inhalation oxygen and the measured exhalation oxygen.

3. The device according to claim 2 wherein:
the controller is operable to receive an oxygen saturation percentage from the pulse oximeter; and
the controller is operable to determine the hemoglobin level of the patient using the formula:
$$\frac{\begin{matrix}( measured inhalation oxygen - measured exhalation oxygen ) * \\ breath volume\end{matrix}}{oxygen saturation * 1.36} .$$

4. The device according to claim 3 wherein the controller is operable to determine a red blood cell (RBC) count by dividing the determined hemoglobin level by 2.89.

5. The device according to claim 4 further comprising an end tidal carbon dioxide (ETCO2) sensor; wherein the controller is operable to receive an ETCO2 level from the ETC 2 sensor.

6. The device according to claim 5 wherein the controller is operable to determine at least one of a pleth variability index, a perfusion index, blood velocity, blood volume, and cardiac output information based on at least one of the determined hemoglobin level, the ETCO2 level, the oxygen saturation percentage, the breath volume, the breath rate, the oxygen consumption level, and a gas exchange rate.

7. The device according to claim 6 wherein the controller is operable to determine an indication of at least one of acute respiratory distress syndrome (ARDS), sepsis, and septic shock from at least one of the determined hemoglobin level, the ETCO2 level, the oxygen saturation percentage, the breath volume, the breath rate, the oxygen consumption level, and the gas exchange rate.

8. The device according to claim 7 further comprising an alarm;
wherein the controller is operable to determine if data received from any of the first and second oxygen sensors and the first and second volume flow meters is outside a target range; and wherein the controller is operable to operate the alarm responsive to at least one of a sudden drop in the determined hemoglobin level, an indication of ARDS, an indication of sepsis, an indication of septic shock, and an indication of data received from any of the first and second oxygen sensors and the first and second volume flow meters is outside the target range.

9. The device according to claim 2 further comprising a data interface operable to transmit data to a ventilator.

10. The device according to claim 9 wherein the data interface is operable to transmit at least one of hemoglobin level, oxygen saturation, red blood cell count, lung volume, breath rate, and breath volume for display on a monitor comprised by the ventilator.

11. The device according to claim 9 wherein the controller is operable to send instructions to cause the ventilator to change its operation responsive to the determined hemoglobin level.

12. The device according to claim 11 wherein the controller is operable to receive instructions from a user via a remote access device; and wherein the controller is operable to send instructions to cause the ventilator to change its operation responsive to the instruction received by the remote access device.

13. The device according to claim 1 wherein:
the first oxygen sensor and first volume flow meter transmit data to the controller via a first fluid data path; and
the second oxygen sensor and second volume flow meter transmit data to the controller via a second fluid data path.

14. The device according to claim 1 further comprising an interface operable to receive patient statistics.

15. The device according to claim 14 wherein:
the controller is operable to receive patient statistics via the interface; and
the controller is operable to make a recommendation regarding at least one of a change to a ventilator setting and a disease diagnosis responsive to the patient statistics and the determined hemoglobin level.

16. A hemoglobin measuring device comprising
a controller;
a first oxygen sensor:
a second oxygen sensor;
a first volume flow meter;
a second volume flow meter; and
a data interface operable to transmit data to a ventilator;
wherein the controller is operable to receive data from each of the first oxygen sensor, the first volume flow meter, the second oxygen sensor, the second volume flow meter, and a pulse oximeter associated with a patient; and
wherein the controller is operable to determine a measured inhalation oxygen from data received from the first oxygen sensor and the first volume flow meter;
wherein the controller is operable to determine a measure exhalation oxygen from data received from the second oxygen sensor and the second volume flow meter;
wherein the controller is operable to determine a breath volume from data received from the first volume flow meter and the second volume flow meter;
wherein the controller is operable to receive an oxygen saturation percentage from the pulse oximeter;
wherein the controller is operable to determine a hemoglobin level of the patient using the formula:
$$\frac{\begin{matrix}( measured inhalation oxygen - measured exhalation oxygen ) * \\ breath volume\end{matrix}}{oxygen saturation * 1.36} ;$$
and
wherein the data interface is operable to transmit at least one of hemoglobin level, oxygen saturation, red blood cell count, lung volume, breathing rate, and breath volume for display on a monitor comprised by the ventilator.

17. The device according to claim 16 wherein the controller is operable to determine a red blood cell (RBC) count by dividing the hemoglobin level by 2.89.

18. The device according to claim 16 wherein the controller is operable to send instructions to cause the ventilator to change its operation responsive to the determined hemoglobin level.

19. The device according to claim 18 further comprising a remote access device; wherein the controller is operable to receive instructions from a user via the remote access device; and wherein the controller is operable to send instructions to cause the ventilator to change its operation responsive to the instruction received by the remote access device.

20. The device according to claim 16 further comprising an interface operable to receive patient statistics; wherein the controller is operable to receive patient statistics via the interface; and wherein the controller is operable to make a recommendation regarding at least one of a change to a ventilator setting and a disease diagnosis responsive to the patient statistics and the determined hemoglobin level.
